(19) United States
(12) Patent Application Publication
(10) Pub. No.: US 2025/0332438 A1
(43) Pub. Date: Oct. 30, 2025

(54) TREATMENT SENSING DEVICE

(71) Applicant: InnoLux Corporation, Miao-Li County (TW)

(72) Inventors: Chun-Kai LEE, Miao-Li County (TW); Cheng-Hsu CHOU, Miao-Li County (TW); Yi-Shu CHEN, Miao-Li County (TW); Jih-Ping LIN, Miao-Li County (TW); Fang-Iy WU, Miao-Li County (TW)

(21) Appl. No.: 19/089,470

(22) Filed: Mar. 25, 2025

(30) Foreign Application Priority Data
Apr. 25, 2024 (CN) 202410503505.3

(51) Int. Cl. A61N 5/06; G01J 1/44; H10F 39/00; H10F 39/90; H10H 29/32

(52) U.S. Cl. CPC A61N 5/0616; G01J 1/44; H10F 39/8037; H10F 39/90; H10H 29/32; A61N 2005/0628; A61N 2005/0652

(57) ABSTRACT

A treatment sensing device includes a substrate, a diode, a first transistor and a second transistor. The diode is disposed on the substrate and includes a first end. The first transistor is disposed on the substrate and includes a first end and a second end, wherein the first end of the first transistor is electrically connected to the first end of the diode. The second transistor is disposed on the substrate and includes a first end and a second end, wherein the first end of the second transistor is electrically connected to the first end of the diode. When the diode is in a light-emitting mode, the second end of the first transistor provides a positive voltage to the diode. When the diode is in the sensing mode, the second end of the second transistor provides a ground voltage or a negative voltage to the diode.

### CROSS-REFERENCE TO RELATED APPLICATIONS

This application claims the benefits of the Chinese Patent Application Serial Number 202410503505.3, filed on Apr. 25, 2024, the subject matter of which is incorporated herein by reference.

### BACKGROUND

### Field of the Disclosure

The present disclosure relates to a sensing device and, more particularly, to a treatment sensing device.

### Description of Related Art

For the prior integrated device with light-emitting components and photosensitive components, more than two components, such as a light-emitting diode (LED) and a photodiode (PD) are required, and thus, as the components are arranged in an array, the resolution (spacing) of the components will be inevitably affected. For example, placing a photodiode between two light-emitting diodes will inevitably increase the distance between the two light-emitting diodes thereby affecting uniformity and light intensity and, vice versa, the resolution sensed by the photodiodes will also be affected by the light-emitting diode. In addition, the overall packaging of two different sizes of components is also a big challenge.

In order to solve the above problems, currently, a direct solution is to select small-sized components and to reduce the spacing between components. However, this solution will be limited by component size and process limits, which cannot meet actual needs.

Therefore, it is necessary to provide a novel sensing device to alleviate and/or obviate the aforementioned problems.

### SUMMARY

The present disclosure provides a treatment sensing device, which includes: a substrate; a diode disposed on the substrate and provided with a first end; a first transistor disposed on the substrate and provided with a first end and a second end, wherein the first end of the first transistor is electrically connected to the first end of the diode; and a second transistor disposed on the substrate and provided with a first end and a second end, wherein the first end of the second transistor is electrically connected to the first end of the diode, wherein, when the diode is in a light-emitting mode, the second end of the first transistor provides a positive voltage to the diode, wherein, when the diode is in a sensing mode, the second end of the second transistor provides a ground voltage or a negative voltage to the diode.

The present disclosure further provides a treatment sensing device, which includes: a substrate; a diode disposed on the substrate and provided with a first end; a first transistor disposed on the substrate and provided a first end and a second end; a second transistor disposed on the substrate and provided with a first end and a second end; and a fourth transistor disposed on the substrate and provided with a first end and a second end, wherein the first end of the first transistor is electrically connected to the first end of the fourth transistor, the first end of the second transistor is electrically connected to the first end of the fourth transistor, the second end of the fourth transistor is electrically connected to the first end of the diode, wherein, when the diode is in a light-emitting mode, the second end of the first transistor provides a positive voltage to the diode through the fourth transistor, wherein, when the diode is in a sensing mode, the second end of the second transistor provides a ground voltage or a negative voltage to the diode through the fourth transistor.

Other novel features of the disclosure will become more apparent from the following detailed description when taken in conjunction with the accompanying drawings.

### BRIEF DESCRIPTION OF DRAWINGS

FIG. 1A is a schematic diagram showing the structure of the treatment sensing device according to an embodiment of the present disclosure;

FIG. 1B is a cross-sectional view of the treatment sensing device of FIG. 1A along line segment A-A′;

FIG. 2A and FIG. 2B are schematic configuration diagrams of the treatment sensing device according to an embodiment of the present disclosure;

FIG. 3A and FIG. 3B are schematic diagrams showing the optimized configuration of the relative positions of the diodes of the treatment sensing device according to an embodiment of the present disclosure;

FIG. 4 is a schematic diagram showing the diode configuration of the treatment sensing device according to an embodiment of the present disclosure;

FIG. 5 is another schematic diagram showing the structure of the treatment sensing device according to an embodiment of the present disclosure;

FIG. 6 shows an equivalent circuit diagram of the treatment sensing device according to an embodiment of the present disclosure;

FIG. 7 shows another equivalent circuit diagram of the treatment sensing device according to an embodiment of the present disclosure; and

FIG. 8 shows yet another equivalent circuit diagram of the treatment sensing device according to an embodiment of the present disclosure.

### DETAILED DESCRIPTION OF EMBODIMENT

The implementation of the present disclosure is illustrated by specific embodiments to enable persons skilled in the art to easily understand the other advantages and effects of the present disclosure by referring to the disclosure contained therein. The present disclosure is implemented or applied by other different, specific embodiments. Various modifications and changes can be made in accordance with different viewpoints and applications to details disclosed herein without departing from the spirit of the present disclosure.

It should be noted that, in the specification and claims, unless otherwise specified, having “one” element is not limited to having a single said element, but one or more said elements may be provided. Furthermore, in the specification and claims, unless otherwise specified, ordinal numbers, such as “first”, “second”, etc., used herein are intended to distinguish elements rather than disclose explicitly or implicitly that names of the elements bear the wording of the ordinal numbers. The ordinal numbers do not imply what order an element and another element are in terms of space, time or steps of a manufacturing method.

In the entire specification and the appended claims of the present disclosure, certain words are used to refer to specific components. Those skilled in the art should understand that electronic device manufacturers may refer to the same components by different names. The present disclosure does not intend to distinguish those components with the same function but different names. In the claims and the following description, the words “comprise”, “include” and “have” are open type language, and thus they should be interpreted as meaning “including but not limited to . . . ”. Therefore, when the terms “comprise”, “include” and/or “have” are used in the description of the present disclosure, they specify the existence of corresponding features, regions, steps, operations and/or components, but do not exclude the existence of one or more corresponding features, regions, steps, operations and/or components.

In the description, the terms “almost”, “about”, “approximately” or “substantially” usually means within 10%, 5%, 3%, 2%, 1% or 0.5% of a given value or range. The quantity given here is an approximate quantity; that is, without specifying “almost”, “about”, “approximately” or “substantially”, it can still imply the meaning of “almost”, “about”, “approximately” or “substantially”. In addition, the term “range of the first value to the second value” or “range between the first value and the second value” indicates that the range includes the first value, the second value, and other values in between.

Unless otherwise defined, all terms (including technical and scientific terms) used here have the same meanings as commonly understood by those skilled in the art of the present disclosure. It is understandable that these terms, such as those defined in commonly used dictionaries, should be interpreted as having a meaning consistent with the relevant technology and the background or context of the present disclosure, rather than in an idealized or excessively formal interpretation, unless specifically defined.

In addition, relative terms such as “below” or “bottom”, and “above” or “top” may be used in the embodiments to describe the relationship between one component and another component in the drawing. It can be understood that, if the device in the drawing is turned upside down, the components described on the “lower” side will become the components on the “upper” side. When the corresponding member (such as a film or region) is described as “on another member”, it may be directly on the other member, or there may be other members between the two members. On the other hand, when a member is described as “directly on another member”, there is no member between the two members. In addition, when a member is described as “on another member”, the two members have a vertical relationship in the top view direction, and this member may be above or below the other member, while the vertical relationship depends on the orientation of the device.

In the present disclosure, the measurement method of thickness may be obtained by using an optical microscope, and the thickness may be obtained by measuring the cross-sectional image in an electron microscope, but it is not limited thereto. In addition, any two values or directions used for comparison may have certain errors. If the first value is equal to the second value, it implies that there may be an error of about 10% between the first value and the second value. If the first direction is perpendicular to the second direction, the angle between the first direction and the second direction may be in a range of 80 to 100 degrees. If the first direction is parallel to the second direction, the angle between the first direction and the second direction may be in a range of 0 to 10 degrees.

It should be noted that the technical solutions provided by the different embodiments described hereinafter may be used interchangeably, combined or mixed to form another embodiment without violating the spirit of the present disclosure.

In one embodiment, the electronic device may include a display device, a backlight device, an antenna device, a sensing device, a tiled device or a treatment sensing device, but it is not limited thereto. The electronic device may be a bendable or flexible electronic device. The display device may be a non-self-luminous display device or a self-luminous display device. The antenna device may be a liquid crystal type antenna device or a non-liquid crystal type antenna device, and the sensing device may be a sensing device for sensing capacitance, light, thermal energy or ultrasonic waves, but it is not limited thereto. In the present disclosure, the electronic device may include electronic components, and the electronic components may include passive components and active components, such as capacitors, resistors, inductors, diodes, transistors, and the like. The diodes may include light emitting diodes or photodiodes. The light emitting diodes may, for example, include organic light emitting diodes (OLEDs), sub-millimeter light emitting diodes (mini LEDs), micro light emitting diodes (micro LEDs) or quantum dot light emitting diodes (quantum dot LEDs), but it is not limited to. The tiled device may be, for example, a tiled display device or a tiled antenna device, but it is not limited thereto. It is noted that the electronic device may be any permutation and combination of the aforementioned, but it is not limited thereto. In the following description, a treatment sensing device is used as an electronic device to illustrate the content of the disclosure, but the present disclosure is not limited thereto.

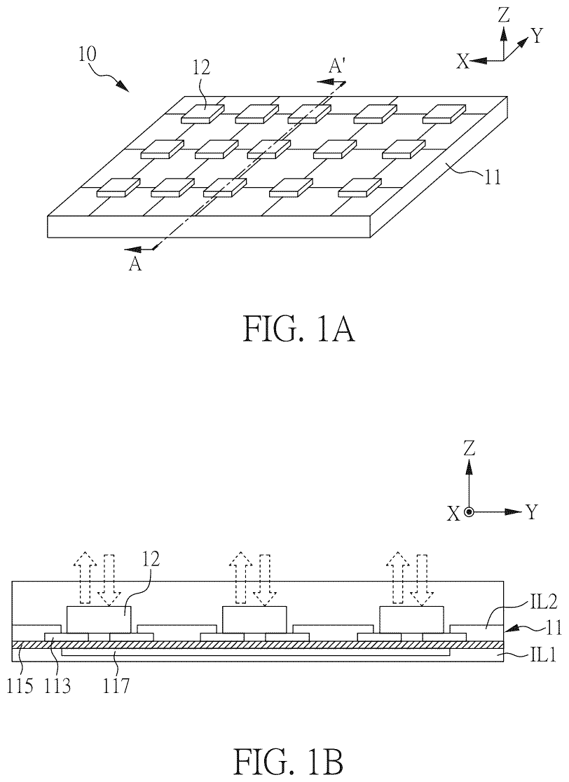

With reference to FIG. 1A and FIG. 1B, FIG. 1A is a schematic diagram showing the structure of the treatment sensing device according to an embodiment of the present disclosure, and FIG. 1B is a cross-sectional view of the treatment sensing device of FIG. 1A along line segment A-A′. As shown, the treatment sensing device 10 includes a substrate 11 and a plurality of diodes 12. The plurality of diodes 12 are, for example, arranged in an array on the substrate 11. The diodes 12 may be arranged on a base material 115 of the substrate 11 through bonding pads 113, the base material 115 is provided with traces 117, and the traces 117 may be electrically connected to the diodes 12 through the bonding pads 113, whereby the diodes 12 may be driven for performing sensing and/or treatment. The base material 115 may be provided with an insulating layer IL1 and an insulating layer IL2 on both sides, at least part of the traces 117 may be disposed between the insulating layer IL1 and the base material 115, and at least part of the bonding pads 113 may be disposed between the insulating layer IL2 and the base material 115. The aforementioned substrate 11 may include a rigid substrate, a flexible substrate, or a combination thereof. For example, the material of the substrate 11 may include glass, quartz, sapphire, ceramic, polycarbonate (PC), polyimide (PI), polyethylene terephthalate (PET), other suitable substrate materials, or a combination thereof, but it is not limited thereto. The aforementioned diode 12 may be, for example, but not limited to, a sub-millimeter light-emitting diode (mini-LED), a micro light-emitting diode (micro-LED), or other suitable diode components. When a forward bias voltage is applied to the diode 12, the diode 12 is in a normal working state to emit light, wherein the forward bias voltage may be a positive voltage. When a reverse bias voltage is applied to the diode 12, since the diode 12 under reverse bias has the photoelectric characteristic that generates current in response to the intensity of external light, the diode 12 under reverse bias may be used as a photodiode (photosensitive component), wherein the reverse bias voltage may be a ground voltage or a negative voltage. The aforementioned insulating layer IL1 and insulating layer IL2 may include appropriate insulating materials, and the insulating layer IL1 and insulating layer IL2 may also be ink, but the present disclosure is not limited thereto.

Therefore, in the treatment sensing device 10 of the present disclosure, the diode 12 to which forward bias voltage is applied may emit light and serve as a light-emitting diode (light-emitting component), and the diode 12 to which reverse bias voltage is applied may be used as a photodiode (photosensitive component). Accordingly, by combining the photosensitive characteristics of the diode 12 under reverse bias and the light-emitting characteristics of the diode 12 under forward bias, a light detection module may be formed, with which the treatment sensing device 10 may be made into a flexible diode patch to perform phototherapy and physiological signal measurement. That is, with the treatment sensing device 10 of the present disclosure, a plurality of diodes 12 may be controlled to serve as light-emitting diodes for performing phototherapy, or a portion of a plurality of diodes 12 may be controlled to serve as light-emitting diodes to emit light source to the human body and another portion of the plurality of diodes 12 may be controlled to serve as photodiodes to sense the aforementioned light source reflected from the human body, so as to detect the condition of the human body and, based on the sensing results, control a plurality of diodes 12 to serve as light-emitting diodes for performing appropriate phototherapy. As a result, the treatment sensing device 10 may be provided with a measurement function without affecting the intensity and uniformity of phototherapy. Furthermore, when the diode 12 is a micro-LED that has a relatively small size (for example, approximately 50 μm), and a portion of the diodes 12 are used as light-emitting diodes, the distance between the portion of the diode 12 may not be enlarged by the other portion of the diode 12 serving as photodiodes to affect the light-emitting function.

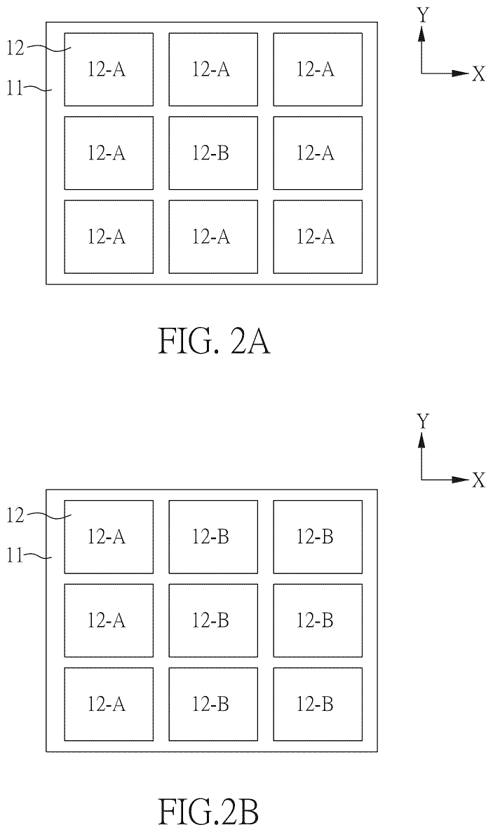

Please refer to FIG. 2A and FIG. 2B, which shows schematic configurations of the treatment sensing device according to an embodiment of the present disclosure, in which the plurality of diodes 12 on the substrate 11 are arranged in an array, and the diodes 12-A serving as light-emitting diodes and the diodes 12-B serving as photodiodes are configured to be one-to-one, many-to-one, or many-to-many, depending on the signal-to-noise ratio under the measurement position and shape. In FIG. 2A, the diodes 12-A serving as light-emitting diodes and the diode 12-B serving as a photodiode are configured to be many-to-one. Since the number of light-emitting diodes is relatively large, it is suitable for the treatment sensing application in which photodiodes require more light sources, such as treatment and sensing of thick skin tissue. In FIG. 2B, the diodes 12-A serving as a light-emitting diodes and the diodes 12-B serving as photodiodes are configured to be one-to-two. Since the number of light-emitting diodes is relatively small, it is suitable for the treatment sensing application in which photodiodes require less light sources. In addition, in order to increase the current generated by the diode 12-B due to photosensitivity, the diodes 12-B may be connected in series and/or in parallel to increase the photosensitive area and thus increase the photocurrent. Moreover, the reverse bias voltage applied to the diode 12-B may increase the photo responsivity of the diode 12-B. In one embodiment, the reverse bias voltage ranges from 0 V to −5 V. In another embodiment, the reverse bias voltage ranges from −2 to −4 V.

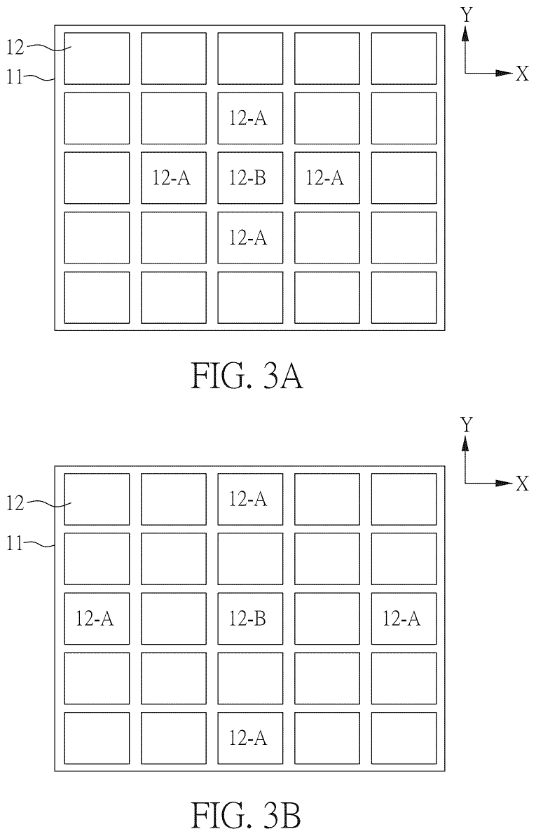

Please refer to FIG. 3A and FIG. 3B, which schematically shows the configuration of the relative positions of the diodes of the treatment sensing device according to another embodiment of the present disclosure, wherein the diode 12 not marked as 12-A or 12B is an inactive component. In FIG. 3A, the diodes 12-A serving as the light-emitting diodes are directly disposed next to the diode 12-B serving as the photodiode, with a relatively small distance therebetween. In FIG. 3B, the diodes 12-A serving as the light-emitting diodes and the diode 12-B serving as the photodiode are separated by at least one inactive diode 12, and thus there is relatively large distance therebetween. The signal-to-noise ratio of the phototherapy and physiological signal measurement module formed by diodes 12-A and diodes 12-B can be optimized by the distance between the aforementioned diode 12-A and diode 12-B, for example but not limited to several millimeters (mm). In one embodiment, when the diode 12 (12A or 12B) is a sub-millimeter light-emitting diode (mini-LED) with a wavelength of 660 nm, the distance between the diode 12-A and the diode 12-B may be 2˜3 mm to have better signals, but the present disclosure is not limited thereto.

Furthermore, the treatment sensing device of the present disclosure may be applied to measure the size and position of wounds. That is, the size and position of wounds on the human body may be measured through image recognition. For example, the diodes 12-B serving as photodiodes that receive weak light source correspond to the wound area of the human body. Therefore, when performing phototherapy, the diodes 12 corresponding to the position of the wound area are all used as light-emitting diodes. It is noted that, the diode 12-A serving as a light-emitting diode herein may be the diode 12-B serving as a photodiode during image recognition, and is instead applied with a forward bias voltage during phototherapy to serve as a light-emitting diode.

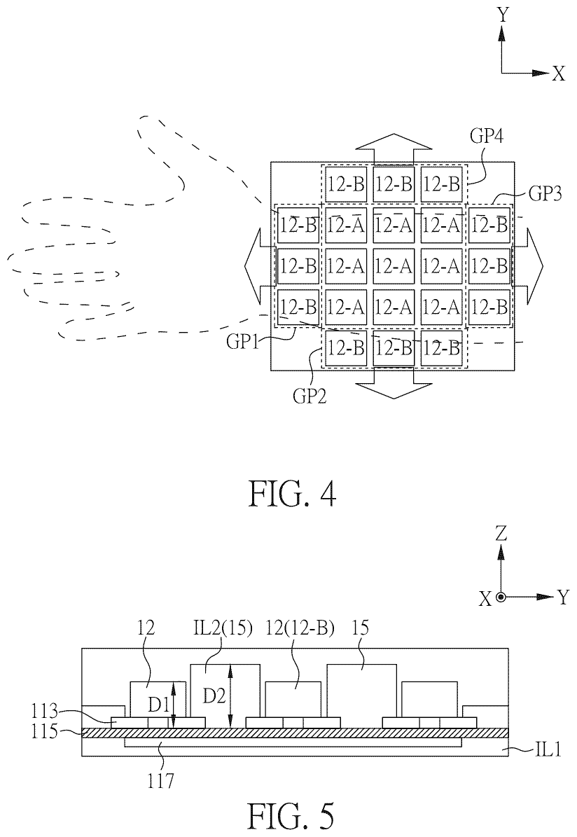

FIG. 4 is a schematic diagram showing the diode configuration of the treatment sensing device according to an embodiment of the present disclosure. In FIG. 4, the diodes 12-A serving as the light-emitting diodes are disposed in the center of the treatment sensing device (flexible diode patch), and the diodes 12-B serving as photodiodes are disposed around or at the four corners of the treatment sensing device. As a result, when the treatment sensing device is placed on the wrist of a human body, the photodiodes around the treatment sensing device 10 are divided into four groups GP1˜GP4 to capture signals so as to determine whether there is measurable heart rate information at the flexible diode patch (that is, artery position), wherein, due to the radial artery of the human body extends along the length direction of the arm, the photodiodes in the groups GP1 and GP3 may measure better heart rate signals.

FIG. 5 is another schematic diagram showing the structure of the treatment sensing device according to an embodiment of the present disclosure, and please refer to FIG. 1B at the same time. In this embodiment, by adjusting the thickness and color of the insulating layer IL2, which may be an ink photoresist 15 in this embodiment, adjacent to the diode 12 on the base material 115, it is able to reduce the noise of the diode 12-B serving as a photodiode caused by the external light so as to increase the signal-to-noise ratio. For example, the height of the diode 12-B may be smaller than the height of the ink photoresist 15. Specifically, the height of the diode 12-B is, for example, the maximum distance D1 from the upper surface of the base material 115 to the top surface of the diode 12-B, and the height of the ink photoresist 15 is, for example, the maximum distance D2 from the upper surface of the base material 115 to the top surface of the ink photoresist 15, so that the maximum distance D1 is smaller than the maximum distance D2. In other embodiments, the ink photoresist 15 may be a black ink photoresist 15, thereby reducing the lateral light leakage of the diode 12-B and the influence of ambient light.

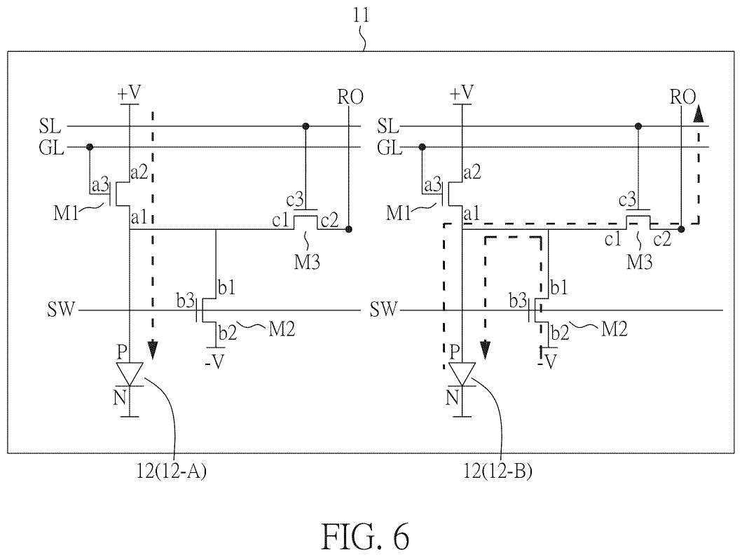

With reference to FIG. 6, which shows an equivalent circuit diagram of a treatment sensing device according to an embodiment of the present disclosure, the treatment sensing device of this embodiment is of active driving type. For convenience of explanation, FIG. 6 only shows two diodes 12 (12-A and 12-B) arranged on the substrate 11 and the driving-related transistors, in which each diode 12 is electrically connected to a first transistor M1, a second transistor M2 and a third transistor M3 to achieve a light-emitting mode or a sensing mode. Since the connection of each diode 12 and its driving-related transistors is the same, only one diode 12 (12-A) and its driving-related transistors will be described in the following. As shown, the diode 12-A has a first end P and a second end N, the first transistor M1 has a first end a1, a second end a2 and a control end a3, the second transistor M2 has a first end b1, a second end b2 and a control end b3, and the third transistor M3 has a first end c1, a second end c2 and a control end c3. The first end a1 of the first transistor M1 is electrically connected to the first end P of the diode 12-A, the second end a2 of the first transistor M1 is electrically connected to the forward bias voltage, the control end a3 of the first transistor M1 is electrically connected to the gate line GL, the first end b1 of the second transistor M2 is electrically connected to the first end P of the diode 12-A, the second end b2 of the second transistor M2 is electrically connected to the reverse bias voltage, the control end b3 of the second transistor M2 is electrically connected to the switch line SW, the first end c1 of the third transistor M3 is electrically connected to the first end b1 of the second transistor M2, the second end c2 of the third transistor M3 is electrically connected to the readout end RO, the control end c3 of the third transistor M3 is electrically connected to the selection line SL, and the second end N of the diode 12-A is grounded, wherein the forward bias voltage is a positive voltage +V, and the reverse bias voltage is a ground voltage or a negative voltage −V.

With the above circuit connection, the diode 12 may be operated to achieve a light-emitting mode or a sensing mode by driving the gate line GL and the selection line SL. When the diode 12 is in the light-emitting mode, the second end a2 of the related first transistor M1 provides a positive voltage +V to the diode 12 and, when the diode 12 is in a sensing mode, the second end b2 of the related second transistor M2 provides a ground voltage or a negative voltage −V to the diode 12. In one embodiment, the voltage connected to the second end b2 of the second transistor M2 ranges from 0 V to −5 V. In another embodiment, the voltage connected to the second end b2 of the second transistor M2 ranges from −2 V to −4 V. In detail, in the following description, the diode 12-A is exemplified by a light-emitting diode and diode 12-B is exemplified by a photodiode. As shown, for the diode 12-A and its related transistors, the selection line SL turns off the third transistor M3, the switch line SW turns off the second transistor M2, and the gate line GL turns on the first transistor M1 so as to provide the positive voltage +V on the second end a2 of the first transistor M1 to the diode 12-A thereby enabling the diode 12-A to be forward biased to serve as a light-emitting diode. Furthermore, for the diode 12-B and its associated transistors, the gate line GL turns off the first transistor M1 and the switch line SW turns on the second transistor M2 so as to provide the ground voltage or negative voltage −V on the second end b2 of the second transistor to the diode 12-B, thereby enabling the diode 12-B to be reverse biased to serve as a photodiode. At this moment, the selection line SL turns on the third transistor M3 so that the sensing result of the photodiode may be read out by the second end c2 (that, readout end RO) of the third transistor M3.

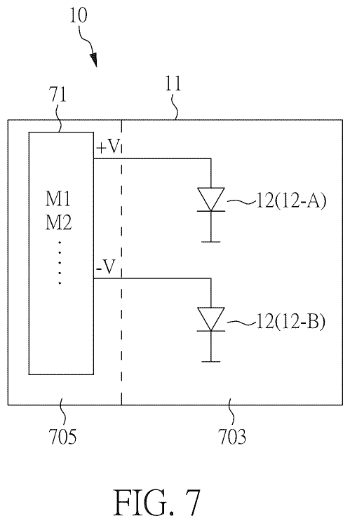

FIG. 7 shows another equivalent circuit diagram of the treatment sensing device according to an embodiment of the present disclosure. The treatment sensing device of this embodiment is of passive driving type. For convenience of explanation, FIG. 7 only shows two diodes 12 (12-A, 12-B) arranged on the substrate 11. In the following description, the diode 12-A is exemplified by a light-emitting diode and the diode 12-B is exemplified by a photodiode. In the treatment sensing device 10 of passive driving type, the treatment sensing device 10 includes an inner area 703 and an outer area 705; for example, the substrate 11 of the treatment sensing device 10 may be divided into an inner area 703 and an outer area 705. The diodes 12-A and 12-B are arranged in the inner area 703 and are driven by an external circuit 71 arranged in the outer area 705. The external circuit 71 may, for example, include the driving circuit composed of the first transistor M1, the second transistor M2, the third transistor M3, etc. as shown in FIG. 6. Therefore, the external circuit 71 may apply a positive voltage +V to the diode 12-A to cause the diode 12-A to serve as a light-emitting diode, and the external circuit 71 may apply a ground voltage or negative voltage −V to the diode 12-A to cause the diode 12-A to serve as a photodiode, wherein the external circuit 17 may be an integrated circuit chip, a gate driver, a data driver, etc., but the present disclosure is not limited thereto.

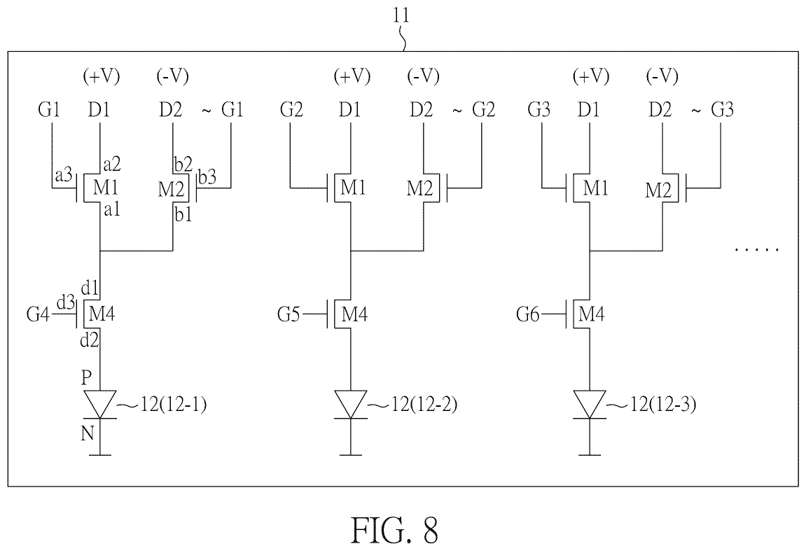

FIG. 8 shows yet another equivalent circuit diagram of the treatment sensing device according to an embodiment of the present disclosure. For convenience of explanation, FIG. 8 only shows three diodes 12 (12-1, 12-2, 12-3) arranged on the substrate 11 and the driving-related transistors, in which each diode 12 is operated with a first transistor M1, a second transistor M2 and a fourth transistor M4 to achieve a light-emitting mode or a sensing mode. Since the connection of each diode 12 and its driving-related transistors is the same, only one diode 12 (12-1) and its driving-related transistors will be described below to illustrate its circuit connection. As shown, the diode 12-1 has a first end P and a second end N, the first transistor M1 has a first end a1, a second end a2 and a control end a3, the second transistor M2 has a first end b1, a second end b2 and a control end b3, and the fourth transistor M4 has a first end d1, a second end d2 and a control end d3. The first end a1 of the first transistor M1 is electrically connected to the first end d1 of the fourth transistor M4, the second end a2 of the first transistor M1 is electrically connected to the data line D1, and the control end a3 of the first transistor M1 is electrically connected to the gate line G1. The first end b1 of the second transistor M2 is electrically connected to the first end d1 of the fourth transistor M4, the second end b2 of the second transistor M2 is electrically connected to the data line D2, and the control end b3 of the second transistor M2 is electrically connected to the inverted gate line ˜G1, the second end d2 of the fourth transistor M4 is electrically connected to the first end P of the diode 12-1, the control end d3 of the fourth transistor M4 is electrically connected to the gate line G4, and the second end N of the diode 12-1 is grounded.

With the above circuit connection, under the control of two gate lines (G1 and ˜G1) that are inverted to each other, the first end P of the diode 12-1 may be connected to the data line D1 through the fourth transistor M4 and the third transistor M4, or connected to the data line D2 through the fourth transistor M4 and the second transistor M2, wherein the data line D1 may, for example, have a positive voltage +V, and the data line D2 may, for example, have a ground voltage or a negative voltage −V. Therefore, with the two gate lines (G1 and ˜G1) that are inverted to each other, the diode 12-1 may be controlled to be forward biased or reverse biased for serving as a light-emitting diode or photodiode. Similarly, with the two gate lines (G2 and ˜G2) that are inverted to each other in FIG. 8, the diode 12-2 may be controlled to be forward biased or reverse biased for serving as a light-emitting diode or a photodiode and, with the two gate lines (G3 and ˜G3) that are inverted to each other in FIG. 8, the diode 12-3 may be controlled to be forward biased or reverse biased for serving as a light-emitting diode or a photodiode. Furthermore, in this embodiment, the fourth transistor M4 related to the diode 12-1 may be controlled to be turned on or off by the gate line G4 thereby selecting whether to activate the diode 12-1. Similarly, the fourth transistor M4 related to the diode 12-2 may be controlled to be turned on or off by the gate line G5 thereby selecting whether to activate the diode 12-2, and the fourth transistor M4 related to the diode 12-3 may be controlled by turned on or off by the gate line G6 thereby selecting whether to activate the diode 12-3. Accordingly, by activating the diodes 12-1, 12-2, 12-3 at suitable time, it not only saves electricity, but also improves the effect of treatment.

In addition, with regard to the treatment sensing device 10 shown in FIG. 8, and with reference to FIG. 7, the diodes 12-1, 12-2 and 12-3 may be disposed in the inner area 703 of the treatment sensing device 10, and the first transistor M1, the second transistor M2, the fourth transistor M4, etc. for driving may be disposed in the outer area 705 of the treatment sensing device 10, thereby forming a treatment sensing device 10 of passive driving type.

The features of various embodiments of the present disclosure may be mixed and matched as long as they do not violate the spirit of the disclosure or conflict with each other.

The aforementioned specific embodiments should be construed as merely illustrative, and not limiting the rest of the present disclosure in any way.

1. A treatment sensing device, comprising:
a substrate;
a diode disposed on the substrate and provided with a first end;
a first transistor disposed on the substrate and provided with a first end and a second end, wherein the first end of the first transistor is electrically connected to the first end of the diode; and
a second transistor disposed on the substrate and provided with a first end and a second end, wherein the first end of the second transistor is electrically connected to the first end of the diode,
wherein, when the diode is in a light-emitting mode, the second end of the first transistor provides a positive voltage to the diode,
wherein, when the diode is in a sensing mode, the second end of the second transistor provides a ground voltage or a negative voltage to the diode.

2. The treatment sensing device as claimed in claim 1, wherein the second end of the second transistor is connected to a voltage in a range of 0 V to −5 V.

3. The treatment sensing device as claimed in claim 2, wherein the second end of the second transistor is connected to a voltage in a range of −2 V to −4 V.

4. The treatment sensing device as claimed in claim 1, further comprising a third transistor disposed on the substrate, a first end of the third transistor being electrically connected to the first end of the second transistor.

5. The treatment sensing device as claimed in claim 4, wherein, when the diode serves as a light-emitting diode, the first transistor is turned on, and the second transistor and the third transistor are turned off.

6. The treatment sensing device as claimed in claim 5, wherein, when the diode serves as a photodiode, the first transistor is turned off, and the second transistor and the third transistor are turned on.

7. The treatment sensing device as claimed in claim 1, wherein the treatment sensing device includes an inner area and an outer area, the diode is disposed in the inner area, and the first transistor and the second transistor are disposed in the outer area.

8. The treatment sensing device as claimed in claim 1, wherein the diode is provided with a second end connected to ground.

9. The treatment sensing device as claimed in claim 1, further comprising an ink photoresist disposed adjacent to the diode, wherein a height of the diode is smaller than a height of the ink photoresist.

10. The treatment sensing device as claimed in claim 1, wherein the ink photoresist is a black ink photoresist.

11. The treatment sensing device as claimed in claim 4, wherein the second end of the first transistor is electrically connected to the positive voltage, and a control end of the first transistor is electrically connected to a gate line.

12. The treatment sensing device as claimed in claim 4, wherein the second end of the second transistor is electrically connected to the ground voltage or the negative voltage, and a control end of the second transistor is electrically connected to a switch line.

13. The treatment sensing device as claimed in claim 4, wherein a second end of the third transistor is electrically connected to a readout end, and a control end of the third transistor is electrically connected to a selection line.

14. The treatment sensing device as claimed in claim 7, wherein the diode is driven by an external circuit arranged in the outer area.

15. A treatment sensing device, comprising:
a substrate;
a diode disposed on the substrate and provided with a first end;
a first transistor disposed on the substrate and provided a first end and a second end;
a second transistor disposed on the substrate and provided with a first end and a second end; and
a fourth transistor disposed on the substrate and provided with a first end and a second end,
wherein the first end of the first transistor is electrically connected to the first end of the fourth transistor, the first end of the second transistor is electrically connected to the first end of the fourth transistor, the second end of the fourth transistor is electrically connected to the first end of the diode, wherein, when the diode is in a light-emitting mode, the second end of the first transistor provides a positive voltage to the diode through the fourth transistor,
wherein, when the diode is in a sensing mode, the second end of the second transistor provides a ground voltage or a negative voltage to the diode through the fourth transistor.

16. The treatment sensing device as claimed in claim 15, wherein the second end of the first transistor is electrically connected to a first data line having the positive voltage, and a control end of the first transistor is electrically connected to a first gate line.

17. The treatment sensing device as claimed in claim 15, wherein the second end of the second transistor is electrically connected to a second data line having a ground voltage or a negative voltage, and a control end of the second transistor is electrically connected to an inverted first gate line.

18. The treatment sensing device as claimed in claim 15, wherein a control end of the fourth transistor is electrically connected to a second gate line.

19. The treatment sensing device as claimed in claim 15, wherein the treatment sensing device includes an inner area and an outer area, the diode is disposed in the inner area, and the first transistor, the second transistor and the fourth transistor are disposed in the outer area.

20. The treatment sensing device as claimed in claim 15, wherein the diode is provided with a second end connected to ground.
